(19) United States
(12) Patent Application Publication
(10) Pub. No.: US 2014/0213999 A1
(43) Pub. Date: Jul. 31, 2014

(54) Biological Chamber Interbody Spinal Fusion

(71) Applicant: Olympus Biotech Corporation, Hopkington, MA (US)

(72) Inventor: Christophe Geisert, Newtonville, MA (US)

(73) Assignee: Olympus Biotech Corporation, Hopkington, MA (US)

(21) Appl. No.: 13/751,440

(22) Filed: Jan. 28, 2013

(51) Int. Cl. A61M 5/00

(52) U.S. Cl. CPC A61M 5/00
USPC 604/506

(57) ABSTRACT

Embodiments of the current invention provide minimally invasive methods for interbody fusion. In one aspect, the invention relates to a method of treating a patient that includes the steps of introducing a channel-forming instrument into a first vertebra, forming a channel through an endplate of the first vertebra and into a nucleus of an adjacent intervertebral disc without penetrating an annulus of the disc, and supplying a therapeutic agent to the nucleus of the disc via the channel.

### TECHNICAL FIELD

The invention relates to methods of treating patients and, more particularly, to systems and methods for creating a biologic chamber for the treatment of bone and/or spine conditions.

### BACKGROUND

Back pain, particularly lower back pain, is the fifth most common reason for all physician visits in the United States. See Roger Chou et al. Diagnosis and Treatment of Low Back Pain: A Joint Clinical Practice Guideline from the American College of Physicians and the American Pain Society. 147 Annals of Internal Medicine pp. 478-491 (Oct. 2, 2007). Back pain can be managed in some patients with conservative treatments such as exercise, acupuncture, massage or steroid injection, but certain disc conditions such as disc herniation or degeneration, or vertebral conditions such as fracture and spondylolisthesis, require more invasive treatments such as spinal fusion and fixation.

Spinal fusion is a surgical procedure in which two or more vertebrae are fused—permanently bridged, typically by means of a bone graft—to prevent or decrease movement at or around a site of spinal injury or malformation. In a “posterolateral fusion” procedure, bone is grafted between the transverse processes of adjacent vertebrae, while in an “interbody fusion” procedure the intervertebral disc between adjacent vertebrae is removed (termed a “discectomy”) and bone is grafted between the vertebral bodies of those vertebrae, replacing the disc. The fusion of vertebrae can be facilitated by the use of rigid implantable fixation devices such as bone screws, rods and plates, which limit the movement of the vertebrae to be fused relative to one another, or—in the case of interbody fusion—through the use of cages that fit between the adjacent vertebral bodies to contain the bone graft and/or maintain spacing between vertebrae to be fused.

Interbody fusion procedures are typically done using anterior or posterior approaches. In a posterior approach (such as the “posterior lumbar interbody fixation” or “PLIF”) the spine is accessed via a posterior incision which is relatively straightforward for the surgeon. In an anterior approach (as in the “anterior lumbar interbody fixation” or “ALIF”), the approach is through an abdominal incision, which is more complicated for the surgeon. Other approaches, such as transforaminal (TLIF) and “extreme lateral” (XLIF) utilize a lateral or posterolateral access. PLIF and TLIF procedures can sometimes be done by minimally invasive surgical means at lower cost and with fewer complications than fusions done by open surgery. See John C. Lucio et al., Economics of less invasive spinal surgery: an analysis of hospital cost differences between open and minimally invasive instrumented spinal fusion procedures during the perioperative period, 5 Risk Management and Healthcare Policy 65-74 (2012). However, even minimally invasive interbody fusions carry a risk of bleeding and complications—and potentially higher treatment cost per patient—because they involve discectomy and the placement of hardware.

### SUMMARY OF THE INVENTION

Embodiments of the current invention decrease the risks described above by providing minimally invasive methods for interbody fusion in which the intervertebral disc is at least partially preserved, obviating the need for a cage in these procedures.

In one aspect, the invention relates to a method of treating a patient that includes the steps of introducing a channel-forming instrument into a first vertebra, forming a channel through an endplate of the first vertebra and into a nucleus of an adjacent intervertebral disc without penetrating an annulus of the disc, and supplying a therapeutic agent to the nucleus of the disc via the channel. In various embodiments, the therapeutic agent is osteoinductive, osteoconductive, osteogenic, chondrogenic, chondroconductive or chondroinductive. The channel can be formed by inserting the instrument into the first vertebra through an articular process of the first vertebra or into a dorsal-facing surface of the first vertebra, and can extend through an endplate of a second vertebra.

In another aspect, the invention relates to a method of treating a patient that includes forming a multidirectional channel within a first vertebra and providing a therapeutic agent to a region that includes a nucleus of an intervertebral disc that is adjacent to the first vertebra. The multidirectional channel is formed by inserting an instrument into either a dorsal facing surface or an articular process of the first vertebrae, forming a first channel section, and then deflecting the instrument and advancing it to define a second channel section that is angled from and contiguous with the first channel section. In various embodiments, the therapeutic agent is osteoinductive, osteoconductive, osteogenic, chondrogenic, chondroconductive or chondroinductive. In some embodiments, the instrument is repeatedly deflected and advanced to form a plurality of second channel sections that are contiguous with the first channel section or with one-another. The channel extends into a second vertebra in some embodiments, and does not extend into the annulus of the intervertebral disc in other embodiments.

In yet another aspect, the invention relates to a method of treating a patient in need of spinal fusion that includes providing an agent that promotes bone growth to a nucleus of an intervertebral disc without penetrating an annulus of that invertertebral disc. Embodiments of the invention also include forming a channel that extends through the endplates of first and second vertebrae abutting the disc, and further stabilizing the first and second vertebrae by affixing pedicle screws connected by a rod to each of the vertebrae. The agent that promotes bone growth can be osteoinductive, osteoconductive, or osteogenic, and can optionally be a bone morphogenetic protein, insulin-like growth factor-1, fibroblast growth factor, transforming growth factor beta, osteonectin, osteogenin, osteocalcin, pharmaceutical agonists or antagonists for cognate receptors of the foregoing, demineralized bone matrix, bone graft, collagen, calcium phosphate ceramic, chondrocytes, chondroblasts, fibroblasts, osteocytes, osteoblasts, pre-osteoblasts, osteoprogenitor cells, mesenchymal stem cells, embryonic stem cells, induced pluripotent cells, or a mixture of any of the foregoing.

### BRIEF DESCRIPTION OF THE DRAWINGS

In the drawings, like reference characters refer to like features through the different views. The drawings are not necessarily to scale, with emphasis being placed on illustration of the principles of the invention.

FIG. 1 includes a schematic parasagittal view of adjacent vertebrae including a cutaway view of the intervertebral disc.

FIG. 2 includes multiple schematic views of adjacent vertebrae during a procedure according to an embodiment of the present invention.

FIG. 3 includes a schematic transverse cross-section through an intervertebral disc and a parasaggital view of adjacent vertebrae during a procedure according to an embodiment of the present invention.

FIG. 4 includes a schematic parasagittal view of adjacent vertebrae including a cutaway view of the intervertebral disc during a procedure according to an embodiment of the invention.

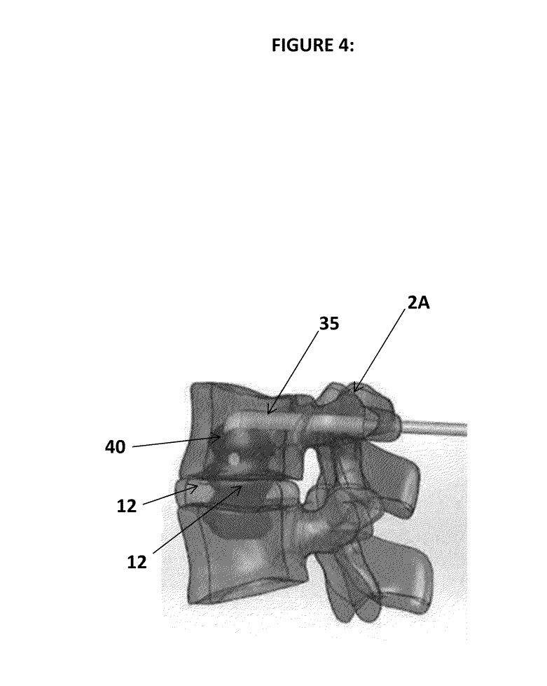

FIG. 5 includes multiple schematic views of adjacent vertebrae during a procedure according to an embodiment of the present invention.

### DETAILED DESCRIPTION OF THE PREFERRED EMBODIMENTS

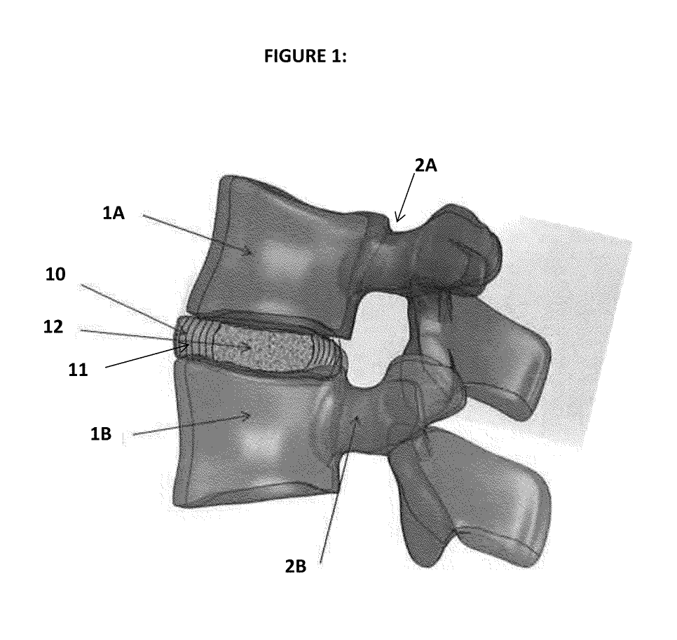

With reference to FIG. 1, in embodiments of the invention, intervertebral fusion between vertebrae 1A, B is achieved without removing disc 10 by providing a therapeutic agent to a region that includes the nucleus pulposis 12 of the disc 10 (hereinafter, the “nucleus”). In preferred embodiments, the annulus fibrosis 11 of the disc 10 (hereinafter, the “annulus”) is left intact and is not punctured along its circumferential edge (i.e. the disc is not punctured from the side). Rather, access to the region including the nucleus 12 is preferably achieved by creating a channel 20 through a portion of a vertebra abutting the disc, such as through the cancellous bone of a first vertebra 1A or 1B. However, different routes may be taken to access the nucleus in other embodiments.

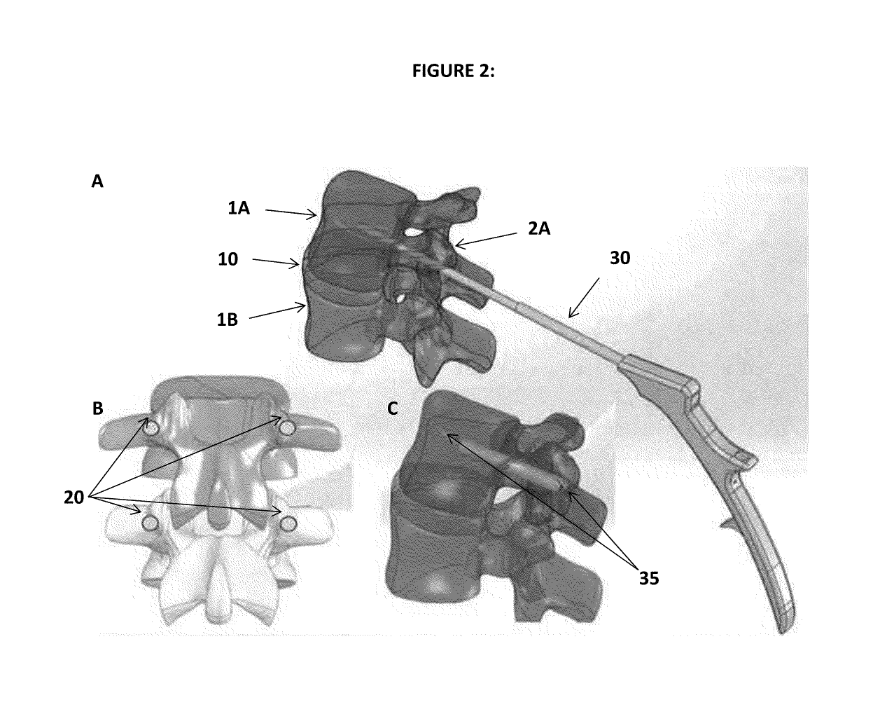

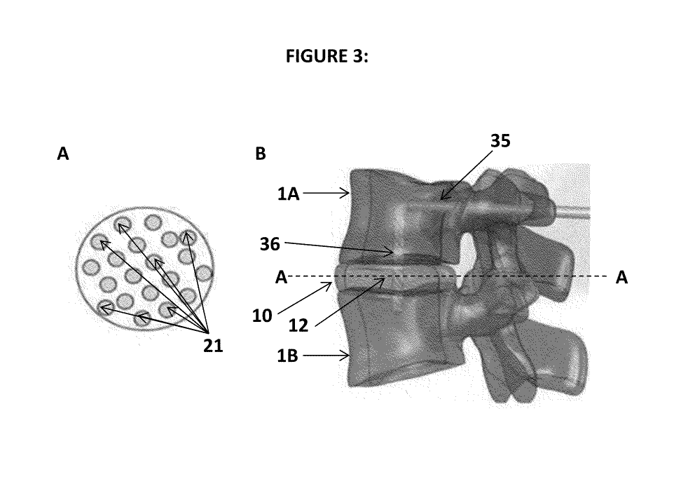

FIGS. 2 and 3 show various schematic views of an exemplary process for accessing the nucleus of a disc according to the invention. FIG. 2B depicts the positioning of holes for pedicle screws in an exemplary pedicle screw placement procedure as currently practiced in the art. In preferred embodiments of the invention, as shown in FIGS. 2A-C, one or more first channel sections 20 are formed through a pedicle 2A of the vertebra 1A into the body of the vertebra 1A. The first channel section or sections 20 preferably serve, after the procedure is complete, as a hole or holes for a pedicle screw or other material. However, placement of a pedicle screw in any first channel section 20 is not required by the invention, and each first channel section 20 can extend through the bone at any suitable angle and along any suitable path to avoid, for example, spinal nerves or the spinal cord.

Any suitable instrument 30 can be used to form the first channel section or sections 20. In some embodiments, the tool 30 is a drill, a shaver, and/or a reamer to penetrate the endplates of the vertebrae 1A, B. In preferred embodiments the instrument 30 includes a tip 35 that is deflectable. Thus, in preferred embodiments, the first section 20 connects to at least one second channel section 21 that extends through the cancellous bone of a vertebra 1 at an angle or along a path that is different than the first channel segment 20 and, most preferably, toward and through the nucleus 12, as shown in FIG. 3B. This arrangement advantageously permits, in preferred embodiments, the first section 20 of the channel to serve as a hole into which a pedicle screw can be anchored as discussed above, thus minimizing the number of holes or incisions that must be made into the spine, and by extension minimizing the risk of damage to the spine or adjacent tissue. However, in other embodiments, even if the first channel section 20 is not subsequently used for a pedicle screw, the first channel section or sections 20 may to extend substantially parallel to or away from the nucleus 12.

In preferred embodiments, a plurality of second channel sections 21 are formed that extend toward (and optionally into and/or through) the nucleus, as shown in FIG. 3A. Any suitable number of second channel sections 21 can be formed within the cancellous bone of the vertebra 1 and/or the nucleus 12, for example 1, 2, 3, 4, 5, 6, 7, 8, 9, 10, 11, 12, 13, 14, 15, 16, 17, 18, 19, 20, or more second channel sections 21 can be formed in various embodiments of the invention.

In preferred embodiments, one or more of the second channel sections 21 extend through the first vertebra 1A, the nucleus 12, and through an endplate and into the cancellous bone of a second vertebra 1B, as shown in FIG. 3B. In other embodiments, the second channel sections 21 extend into the nucleus 12 but do not extend into the second vertebra 1B. The second channel sections 21 preferably define a roughly cylindrical region through the cancellous bone of the vertebra or vertebrae and the nucleus 12 that is sized up to the diameter of the nucleus, generally between 10 and 14 mm in diameter. That is, the diameter of the region shown in cross section in FIG. 3A is generally between about 10 and about 14 mm.

Various relationships may exist between the second channel sections 21 that extend through the nucleus 12 and the channel portion or portions 20 that extend through a pedicle or a dorsal, ventral or lateral surface of the first or second vertebrae 1 A, B. In preferred embodiments, multiple second channel sections 21 extend from a single first channel section 20, minimizing the number of holes that must be drilled through the pedicle and other dorsal, ventral or radial portions of the vertebra 1, and advantageously minimizing the risk of injury to tissues adjacent to the vertebra 1 such as muscles or nerves. However, in other embodiments a 1-to-1 relationship exists such that only one second channel portion 21 extends from any single first channel sections 20.

After one or more of the second channel sections 21 are formed, one or more therapeutic agents are delivered through the first and second channel sections 20, 21 to form a “biologic cloud” 40 that acts as a biological fusion chamber by, preferably, inducing the formation of bone through the nucleus 12. The cloud preferably comprises one or more therapeutic agents that are contained in an area that includes the nucleus 12 and optionally portions of the first and/or second vertebra 1 A, B. The cloud 40 is advantageously prevented from migrating away from the nucleus 12 in preferred embodiments by the intact and un-perforated annulus 11. The cloud 40 preferably promotes osteogenesis in a region between the two vertebrae 1A, B, promoting the formation of new bone and permitting the two vertebrae 1A, B to knit together, while spacing between the vertebrae 1A, B is maintained by the annulus 11 and/or by fixation hardware, as discussed in more detail below.

While any suitable therapeutic agent can be used to form the cloud 40, in preferred embodiments the therapeutic agent is osteoinductive, osteoconductive, osteogenic, chondroinductive, chondroconductive, and/or chondrogenic. Exemplary osteoinductive and/or chondroinductive agents include, without limitation bone morphogenetic proteins (“BMPs”) such as BMP-2, BMP-4, BMP-7, etc., insulin-like growth factor-1 (IGF-1), fibroblast growth factor (FGF), transforming growth factor beta (TGF-beta), osteonectin, osteogenin, osteocalcin, pharmaceutical agonists or antagonists for cognate receptors of the foregoing, etc. Exemplary osteoconductive and/or chondroconductive agents include, without limitation, demineralized bone matrix, bone graft, collagen, and calcium phosphate ceramics. Osteogenic and/or chondrogenic agents include, without limitation, chondrocytes, chondroblasts, fibroblasts, osteocytes, osteoblasts, pre-osteoblasts, osteoprogenitor cells, mesenchymal stem cells, embryonic stem cells, induced pluripotent cells, and the like. Also suitable are bone marrow aspirate stem cells, proteins, and artificial scaffolds.

The therapeutic agent or agents are preferably viscous but flowable, so that it can be flowed through the channel segments 20, 21 to form the cloud 40.

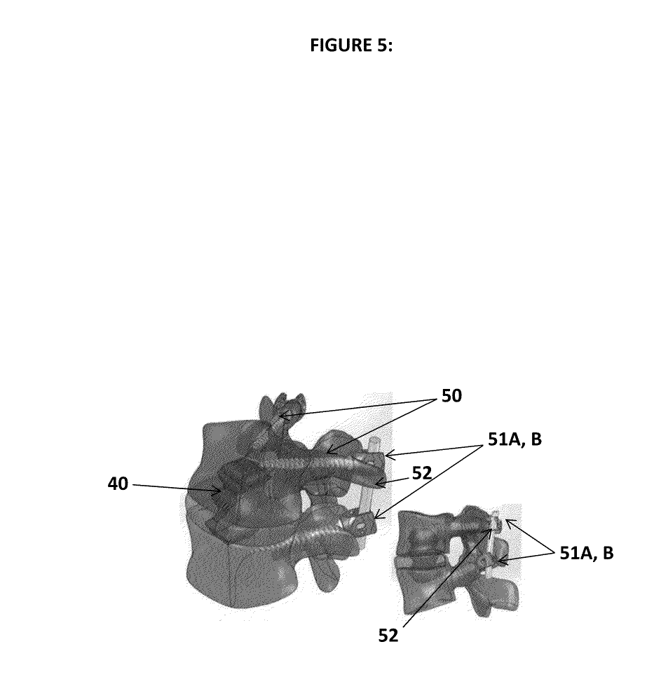

After the cloud is formed, the vertebrae 1A, B are preferably stabilized to permit the vertebrae 1A, B to knit together. In the exemplary embodiment shown in FIG. 5, fixation is achieved by the placement of pedicle screws 50 and a rod 52. The pedicle screws 50 preferably extend through the first channel sections 20, and connect to fixation hardware 51 that is adapted to connect to a rod 52. The rod 52 preferably connects to pedicle screws 51A, B on the same side (e.g. left or right) of the first and second vertebrae 1A, B. In other embodiments, different hardware is used, such as plates, etc. If pedicle screws are not used, it is preferred that bone cement or other suitable material be placed within first channel section(s) 20 to keep the biologic cloud confined in the area to be treated.

Fusion and fixation procedures according to the invention can use any suitable approach. In preferred embodiments, the approach taken is a posterior, postero-lateral or lateral approach, as in PLIF, TLIF and XLIF procedures currently performed in the art. However, anterior approaches can also be taken in accordance with certain embodiments of the invention, in which case the channel portions 20 extend through an anterior or lateral surface of the vertebra 1, rather than a pedicle 2. Procedures according to embodiments of the invention may be done in an open surgical environment, or they may be performed using minimally invasive methods.

Although the examples in this disclosure have focused on procedures that form second channels sections 21 that extend through and downward from a first vertebra superior to a second vertebra, in various embodiments of the invention, the second channel sections 20 can extend through and upward from a first vertebra that is inferior to a second vertebra. By the same token, while this disclosure has focused on interbody spinal fusion and fixation, the methods of the invention can be used in other procedures, such as posterolateral fusion or other procedures to repair a damaged or diseased intervertebral disc. In these embodiments, the therapeutic agent is chondrogenic, chondroinductive, or chondroconductive, and promotes growth or regeneration of the disc. In still other embodiments, methods of the invention are used to deliver therapeutic agents such as chemotherapeutics, therapeutic antibodies, or antiproliferative agents to, e.g. cancerous tissue within a vertebra or an intervertebral disc. Although the examples in this disclosure have focused on fusion and fixation of two vertebrae, any number of vertebrae can be fused by the methods of the invention.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

The terms “first vertebra” and “second vertebra” and the like are meant to refer to adjacent vertebrae that are being fused and, unless otherwise indicated, are not meant to imply any particular anatomical location of the vertebrae or any relationship therebetween.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A method of treating a patient, comprising:
introducing a channel-forming instrument into a first vertebra;
forming a channel with the channel-forming instrument through an endplate of the first vertebra and into a nucleus of an adjacent intervertebral disc without penetrating an annulus of the interverterbral disc; and
providing, via the channel, a therapeutic agent to the nucleus of the intervertebral disc.

2. The method of claim 1, wherein the therapeutic agent is osteoinductive, osteoconductive, or osteogenic.

3. The method of claim 1, wherein the therapeutic agent is chondroinductive, chondroinductive, or chondrogenic.

4. The method of claim 1, wherein the step of forming the channel comprises extending the instrument through an endplate of a second vertebra.

5. The method of claim 1, wherein the instrument is inserted into the first vertebra through an articular process or a dorsal-facing surface of the first vertebra, and forming a channel comprises:
advancing the instrument in a first direction;
deflecting the instrument from the first direction to a second direction; and
advancing the instrument in the second direction through the endplate and into the nucleus.

6. The method of claim 1, further comprising the steps of:
forming a plurality of channels through the endplate of the first vertebra into the nucleus of the intervertebral disc and through an endplate of a second vertebra; and
providing the therapeutic agent to the second vertebra through the plurality of channels.

7. The method of claim 1, further comprising the step of stabilizing the first vertebra.

8. The method of claim 7, wherein stabilizing the first vertebra comprises fixing first and second pedicle screws into the first vertebra and a second vertebra, respectively, wherein the second vertebra is separated from the first vertebra by the intervertebral disc.

9. The method of claim 8, wherein the first and second pedicle screws are connected by a rod.

10. A method of treating a patient comprising the steps of:
forming a multidirectional channel within a first vertebra , wherein the multidirectional channel is formed by (a) inserting an instrument into one of a dorsal-facing surface of the first vertebra and an articular process of the first vertebra and advancing the instrument to form a first channel section, and (b) deflecting the instrument and advancing it to define at least one second channel section contiguous with the first channel, wherein the at least one second channel section is angled relative to the first channel; and
providing, via the multidirectional channel, a therapeutic agent to a region including a nucleus of an intervertebral disc adjacent to the first vertebra.

11. The method of claim 10, wherein the therapeutic agent is osteoinductive, osteoconductive, or osteogenic.

12. The method of claim 10, wherein forming the multidirectional channel includes repeating the steps of
(a) deflecting the instrument and
(b) advancing the instrument
to form a plurality of second channel sections contiguous with the first channel section or with one-another.

13. The method of claim 10, wherein the multidirectional channel extends into the second vertebra.

14. The method of claim 10, wherein the region does not include the annulus of the intervertebral disc.

15. A method of treating a patient in need of spinal fusion, comprising:
providing an agent that promotes bone growth to a nucleus of an intervertebral disc without penetrating an annulus of the intervertebral disc.

16. The method of claim 15, further comprising the steps of forming a channel through first and second endplates of first and second vertebrae abutting the intervertebral disc, respectively, and providing the agent that promotes bone growth to the vertebral bodies via the channel.

17. The method of claim 16, wherein providing the agent that promotes bone growth includes flowing the osteoinductive agent into the channel.

18. The method of claim 16, wherein the step of forming a channel comprises:
inserting an instrument into a cancellous bone of the first vertebra to form a first channel segment, and
deflecting the instrument and advancing it through the endplate of the first vertebra and through the nucleus and into an endplate of the second vertebra to form a second channel segment extending at an angle from the first channel segment.

19. The method of claim 15, further comprising the step of stabilizing first and second vertebrae abutting the intervertebral disc by affixing first and second pedicle screws to the first and second vertebrae, respectively, wherein the first and second pedicle screws are connected to one-another by a rod.

20. The method of claim 15, wherein the agent that promotes bone growth is osteoinductive, osteogenic, or osteoconductive.

21. The method of claim 20, wherein the agent that promotes bone growth includes a bone morphogenetic protein, insulin-like growth factor-1, fibroblast growth factor, transforming growth factor beta, osteonectin, osteogenin, osteocalcin, pharmaceutical agonists or antagonists for cognate receptors of the foregoing, demineralized bone matrix, bone graft, collagen, calcium phosphate ceramic, chondrocytes, chondroblasts, fibroblasts, osteocytes, osteoblasts, pre-osteoblasts, osteoprogenitor cells, mesenchymal stem cells, embryonic stem cells, induced pluripotent cells, or a mixture of any of the foregoing.
